(19) United States
(12) Patent Application Publication
(10) Pub. No.: US 2023/0181751 A1
(43) Pub. Date: Jun. 15, 2023

(54) FORMULATION OF PURE DIMETHOXY CURCUMIN-HUMAN SERUM ALBUMIN AND A PROCESS FOR THE PREPARATION THEREOF

(71) Applicant: SREE CHITRA TIRUNAL INSTITUTE FOR MEDICAL SCIENCES AND TECHNOLOGY, Thiruvananthapuram (IN)

(72) Inventors: Lissy Kalliyana KRISHNAN, Thiruvananthapuram (IN); Renjith Parameswaran NAIR, Thiruvananthapuram (IN); Mejo Chiratteparambil KORAH, Thiruvananthapuram (IN); Deepa SATHEE, Thiruvananthapuram (IN)

(21) Appl. No.: 17/925,385

(22) PCT Filed: May 12, 2021
(86) PCT No.: PCT/IN2021/050453

§ 371 Date: Nov. 15, 2022

(30) Foreign Application Priority Data
May 15, 2020 (IN) 202041020471

(51) Int. Cl. A61K 47/64; A61K 9/19

(52) U.S. Cl. CPC A61K 47/643; A61K 9/19

(57) ABSTRACT

A formulation of pure dimethoxy curcumin-human serum albumin (DMCHSA) comprising a pure di-methoxy curcumin (DMC) bound to human serum albumin (HSA), wherein molar ratio of DMC to HSA is in the range of 3.0-6.0. Further, there is provided a highly soluble and safe intravenous formulation of pure dimethoxy curcumin-human serum albumin retaining proven biological activities and a process for the preparation thereof. Even further, there is provided a process for preparing formulation of pure dimethoxy curcumin-human serum albumin (DMCHSA) by preferential binding of DMC to HSA, which excludes other curcuminoids such as Demethoxy curcumin (DeMC) and Bidemethoxy curcumin (BiDeMC) present in the added chemical, increasing the purity of 80% DMC in starting raw material to >99% in the product DMCHSA.

### FIELD OF THE INVENTION

This invention relates to a formulation of pure dimethoxy curcumin-human serum albumin (DMCHSA).

This disclosure further relates to a highly soluble and safe intravenous formulation of pure dimethoxy curcumin-human serum albumin retaining proven biological activities and a process for the preparation thereof.

The present disclosure is related to field of biomedical science. Specifically, the disclosure is related to a process of preparing a single entity of stably conjugated dimethoxycurcumin (DMC) and human serum albumin (HSA). Further, the present disclosure provides process of increasing the water solubility of DMC from 20 uM to 2 mM for applications in reducing inflammation of human cells at low and nontoxic concentrations and for inducing death of cancer cells at high and safe concentrations.

### BACKGROUND

Science is catching up in news with its stupendous rise in research on turmeric, more significantly the curcumin evolving as the super food in coming years.

Turmeric has been widely used in treating various diseases for at least 2500 years in Asian countries. Dimethoxy curcumin is a low molecular weight lipophilic polyphenol substance which constitutes 2-5% of turmeric powder (Kocaadam et. al 2017). The active compounds in turmeric are typically classified as non-volatile or volatile compounds: Major non-volatile compounds are Curcuminoids, mainly dimethoxy Curcumin (DMC), Demethoxycurcumin (DeMC) and Bidemethoxycurcumin (BiDeMC). Out of these 3 derivatives, the orange-yellow colored DMC is the most active component.

This chemical has gained lot of importance in the scientific literature mainly because of the research over the last two decades showing DMC to be a potent antioxidant, antiinflammatory, anti-proliferative, anti-metastatic, anti-angiogenic, anti-diabetic, hepatoprotective, anti-atherosclerotic, anti-thrombotic, and anti-arthritic agent (Joe et. al, 2004; Dikkal et. al, 2018).

It is remarkably non-toxic but exhibits limited bioavailability. This poor solubility has been highlighted as a major limitation in clinical use.

Curcumin is odor-less powder (melting point 184-186° C.), poorly soluble in water, petroleum ether and benzene; soluble in ethyl alcohol, glacial acetic acid and in propylene glycol; very soluble in acetone, dimethyl sulphoxide (DMSO) and ethyl ether. Absorption spectra of DMC and Curcuminoids are very similar, with their maximum absorption (λ) at 429 nm and 424 nm, respectively.

Chemically it is represented by (1E,6E)-1,7-bis(4-hydroxy-3-methoxyphenyl)hepta-1,6-dienne-3,5-dione. It has a pKa1,pKa2, and pKa3 value of 7.8, 8.5, and 9.0, respectively, for three acidic protons (Bachar Zebib et. al, 2010). Dimethoxy Curcumin (DMC), demethoxycurcumin (DeMC) and Bidemethoxycurcumin (BiDeMC) show very small chemical differences. Even though these variations seem subtle, hydrophobic nature of three curcuminoids is distinct which is in the order of DMC> DeMC >BiDeMC, with DMC being the relatively most hydrophobic among the three curcuminoids.

It is sparingly soluble in water under acidic or neutral conditions. The DMC is unstable undergoing rapid hydrolytic degradation in neutral or alkaline conditions to feruloyl methane and ferulic acid. Since it is insoluble in aqueous medium and has poor stability towards oxidation, light, alkalinity, enzymes and heat, it cannot really be widely used in food and pharmaceutical processing industry. For these reasons, DMC should be protected in certain forms from physical and chemical damage before its pharmaceutical application.

The biological effects of DMC in cellular and animal models is challenging because of its chemical and metabolic instability (Claus Schneider et. al., 2015). The DMC undergoes rapid non-enzymatic degradation in cell culture medium and possibly in vivo as well. It is metabolized primarily by reduction and conjugation after oral administration. Consecutive reduction of the double bonds in the heptadienedione chain results in the formation of di-, tetra-, hexa-, and octa-hydrocurcumin. The reduced metabolites, especially tetra- and hexa-hydrocurcumin, are the largest portion of curcumin metabolites detected. With a few exceptions their biological activities are strongly reduced compared to curcumin.

The DMC degrades quickly at physiological pH but slower when incubated in the presence of serum or with cultured cells. The half-life of DMC is increased by the presence of protein, from a few minutes to 1-2 h (Wang et.al, 1997). More than 70% DMC is degraded in basic conditions, whereas 40-60% degradation was observed in acidic pH conditions at the end of 8h (Ansari et. al, 2016). Even though an ocean of scientific knowledge is available including biochemical pathways of activities, current literature identifies poor solubility and chemical degradation as the major hurdles in the use of curcumin in clinics to treat humans (Dikkal et. al, 2018).

It has been reported that because of low bioavailability, higher concentrations of DMC need to be administered for its effective treatment of the disease. The faster metabolic breakdown of curcumin also has been reported to be a reason for the need of very high dose for achieving significant activity.

Various attempts have been made to overcome the solubility problem, but hardly any attempt has been made to increase the metabolic stability in the physiological system. Nano-formulations, encapsulation in polymers such as polyethylene glycol (PEG), liposomal encapsulations, and conjugations with natural substances such as piperine and several other attempts have been reported (Prasad et al 2014). Different approaches that enable stable drug delivery systems include liposomes, polymeric nanoparticles, dendrimers, and solid lipid nanoparticles. Need for surface engineering to reduce host foreign body responses, whilst maintaining cellular targeting capabilities seems challenging. Non-specific accumulation of synthetic material would seemingly restrict clinical application of nanosystems. This is exemplified by the limited number of nanocarrier-based marketed products.

Consequently, search-for a “super curcumin” without these problems and with efficacy equal to or better than that of native DMC is ongoing. Innovative methods of cancer treatment require new concepts of drug delivery in cancer. There is a vast range of strategies available for drug delivery in cancer. To improve the bioavailability of DMC, numerous approaches have been undertaken. These approaches involve the use of adjuvants that interferes with glucuronidation, liposomal curcumin, nanoparticles, curcumin phospholipid complexes and the use of structural analogues of curcumin. Recent progress in nanoparticle engineering has certainly improved drug targeting, but the results are not as good as expected. This is largely due to the fact that nanoparticles, regardless of how advanced they are, find the target through blood circulation, like the conventional drug delivery systems do. The accumulation of nanoparticles in cells, their toxicity and clearance are issues that are not addressed satisfactorily.

Extensive work has been done on the preparation of water-soluble curcumin by incorporation into various surfactant micellar systems (e.g. sodium dodecyl sulfate, cetylpyridinium bromide, gelatine, polysaccharides, polyethylene glycol and cyclodextrins) have been reported (Humphrey, 1980, Tonnesen, 2002). In another approach, water soluble curcumin complex was synthesized by dissolving and mixing curcumin and gelatin in an aqueous acetic acid solution. All these techniques demand; the release of DMC from the delivery vehicle which poses the major problem of premature drug release, i.e. drug release can occur before reaching the target site. None of the approaches threw light on the molecular mechanisms and experimental data for recommended dosing patterns necessary for destroying the cancer cells.

However, all these interventions do not ensure the efficacious release of DMC, and also fail to address the issue of targeted delivery of curcumin.

Some of the approaches are not hemocompatible. The threshold concentration in the range of 10 µg/ml of free curcumin in plasma resulted in echinocytosis (Stroka et.al, 2013). The authors also demonstrated that the liposomal curcumin caused significant change in the RBC morphology forming enchinocyte with larger RBC volume as well. The study concluded that curcumin and liposomal curcumin cause dose-dependent changes in the shape of RBCs. This effect may represent an early sign of dose-limiting toxicity following intravenous administration. Therefore, hemocompatibility may be an important consideration while modifying the curcumin for achieving solubility and stability.

Exploitation of the natural properties of ligand binding and transport have been utilized for albumin-based drug delivery, with a focus on drug half-life extension. Albumin is an attractive next generation “self” drug delivery approach (Larsen et. al, 2016). Albumin’s inherent transport properties and cellular receptor engagement promotes albumin as a natural medicinal molecule. Serum albumin possesses a unique capability to bind, covalently or reversibly, a great number of various endogenous and exogenous compounds. Several different transport proteins exist in blood plasma but only albumin is able to bind a wide diversity of ligands reversibly with high affinity. On the basis of the amino acid sequence, Brown (1977) proposed a 3-domain model for the protein.

The body is sensitive to the structure of albumin and rapidly eliminates any molecules which have not retained the native structure of albumin. Therefore, only albumin-drug carriers with a molar loading ratio of on average 1 to 4 moles of drug per mole of albumin are physiologically stable and attain half-lives similar to native albumin (Stehle et. al, 1997; Neumann et. al, 2010). The knowledge that the number of molecules bound is critical for maintaining the normal albumin conformation that can be recognized by cell surface receptors, signifies the importance of using therapeutically viable HSA for use as curcumin carrier. Therefore, achieving optimum or improved DMC binding to Human Serum Albumin (HSA) may be useful for conformational stability of the protein to enable drug delivery appropriately.

Song et. al, (2016) prepared curcumin-loaded human serum albumin nanoparticles with or without folate-conjugation, (CM-HSANPs & F-CM-HSANPs) by the chemical conjugation of folate to the surface of the curcumin (CM)-loaded human serum albumin nanoparticles (NPs). They obtained very promising results upon physico-chemical characterization. But the pharmaco-kinetics in animal model showed elimination half-life (t_{½}) values of CM-HSANPs and F-CM-HSANPs 0.36 hours and 0.43 hours, respectively. Another limitation is that they detected high concentrations of free curcumin in the plasma within 5 min of dosing, indicating a burst release that can result in the metabolic degradation of curcumin into inactive forms. All these results suggest that so far the nano formulations of DMC using HSA has not reached a level for use in clinical situations.

In this context, curcumin conjugated with bovine serum albumin (BSA) has been developed which has shown improved solubility by 100-fold and was effective in causing death of cancer cell lines in vitro (LK Krishanan et al, 2014 & Thomas et.al. 2014). Experiments also showed that Dalton’s lymphoma ascites (DLA) cell viability was inhibited by the CMBSA conjugate in a dose dependent manner in vitro, as evidenced by the MTT assay. U.S. Pat. No. 10849983 B2 discloses an albumin-curcumin conjugate for application in cancer therapy, including albumin and curcumin. The study also demonstrated antitumor activity in DLA mouse models (Aravind et.al, 2016). However, when commercially obtained 80% curcuminoid was used for conjugating with BSA, the molar binding ratio was ~0.6 molecule per 1.0 mole of BSA. Therefore, very high concentrations of BSA is required for delivering curcumin for indented biological activity, reducing feasibility of intravenous human use. The use of BSA as drug delivery vehicle in low or high concentrations can cause severe immune response in humans.

Human serum albumin (HSA) offers advantages over BSA for therapeutic use. Both BSA and HSA are similar in terms of molecular weight. However, conformational difference affecting the ligand binding pockets available are different in BSA and HSA.

Albumin is a natural transport protein with multiple ligand binding sites, specific cellular receptors and a long circulatory half-life. Albumin as an attractive candidate for targeted intracellular delivery of drugs through ligand-mediated association seems to be promising.

Interestingly, upon analysing >200 atomic structures of human albumin in complex with various pharmaceuticals and endogenous ligands, it has been reported that small hydrophobic and anionic drugs showed preference for Drug sites 1 and 2, while complex heterocyclic ligands preferred Drug site 3. (Min He et.al 1992, Carter 2010). As ligands and drugs may compete for the same binding sites on albumin, their effect will depend on the presence of competitors, which results in their free fractions relative to the albumin-bound fractions. Occupied binding pockets are known to modulate binding to albumin receptors on the cell surface, and consequently affect bio-distribution, half-life and tissue deposition. While gp60 has been shown to bind native bovine albumin, gp30 and gp18 have been demonstrated to be more universally expressed and to bind modified albumin also and thus act as scavenger receptors that deliver modified albumin to lysosome degradation.

All this knowledge on HSA protein chemistry and the stability of protein upon curcumin binding suggests that a product of curcumin bound HSA may have promising prospects for developing a product for pharmaceutical applications. However, no previous effort has been made to obtain pure DMC as a drug. All the previous attempts focused delivery of curcuminoids.

The teaching that hydrophobic ligands have better affinity for albumin (Mohammadin et. al. 2009) may be a starting point for aspects of this disclosure. Among curcuminoids the most active molecule dimethoxy curcumin (DMC), is also the most hydrophobic one in the mixture. Therefore, the chances of preferential binding of DMC to HSA is high as compared to DesMC and BiDeMC. The chemical nature of the ligand can influence and regulate the accessibility of binding pockets. Regulated but stable ligand binding is crucial for maintenance of three-dimensional conformation of albumin which in turn can influence protein half-life in circulation and also for recognition by receptor to mediate intake into cells.

Therefore, aspects of this disclosure exploited the ability of HSA to compete for more hydrophobic ligands, thereby increasing bioavailability through specific receptor mediated pathways increasing the pharmaceutical viability of DMC for anti-inflammatory effect and anticancer effect.

Clinical need for DMC is beyond its application in cancer treatment. Anti-inflammatory activity of DMC is well established; however, this application is also not exploited due to the poor solubility and stability of pure DMC. Two major life-threatening inflammatory conditions are atherosclerosis and arthritis. The former deals with the vascular system in which endothelial cells (EC) is the inflammatory cell. Whereas the latter deals with the disruption of cartilage due to the action of inflammatory stimuli on chondrocytes thereby affecting the cartilage synthesis adversely. Inflammatory diseases are currently treated with the use of anti-inflammatory drugs, commonly known as non-steroidal anti-inflammatory drugs (NSAIDs). The major objective of these drugs is to block the action of inflammatory cytokines which are responsible for progression of the disease. These drugs act as inhibitors of either the receptors of cytokines or inhibit the signalling pathway. But the major problems associated with these drugs are the risk of numerous side effects they carry upon long term treatments. Therefore, drug delivery systems are considered to be important for reducing the side-effects.

In order to exploit anti-inflammatory and anti-cancerous properties of DMC, intravenous administration may be used for achieving bioavailability at all body parts. Since endothelial cells are readily accessible in the vascular system, intravenous administration of DMC could be beneficial for down regulating inflammatory cytokines. Endothelial cells have shown the presence of albumin receptors on their surface for albumin transport via endocytosis (Tiruppathi et al 1996). Also, arthritic patients have been shown to possess hypo-albuminemia due to excessive demand of albumin at the inflamed joints (Ren et. al, 2013). So, an anti-inflammatory drug-conjugated albumin can cause receptor mediated entry to the inflamed cells.

Furthermore, secreted protein acidic and rich in cysteine (SPARC) has been demonstrated to be over-expressed in both in inflammatory and cancer tissues and it has been suggested that it is responsible for transport and accumulation of albumin at tumour and inflammatory sites. Therefore, development of technology that can promote DMC solubility, stability and ultimate bioavailability are likely to be of great significance in clinical exploitation of this drug for regulation of inflammatory response at dose which may not be cytotoxic to cells. At the same time bioavailability of a higher dose of DMC which is cytotoxic could be useful as anticancer drug and enhance the therapeutic efficacy.

### SUMMARY

It is therefore an object of this disclosure to propose a formulation of pure dimethoxy curcumin-human serum albumin and a process for the preparation thereof.

It is a further object of this disclosure to propose a formulation of pure dimethoxy curcumin-human serum albumin by exploiting the differential hydrophobic property of the curcuminoid molecules in the commercially obtained raw material, facilitating selective binding, also by means of inherent property of albumin to preferentially bind most hydrophobic molecule.

A still further object of this disclosure is to propose a formulation of pure dimethoxy curcumin-human serum albumin which is prepared by the elimination of unbound inactive isoforms/degraded- forms of curcuminoids present in the reaction mixture by molecular sieving technique.

Another object of this disclosure is to propose a formulation of pure dimethoxy curcumin-human serum albumin which has better storage stability by freeze drying into powder form.

Yet another object of this disclosure is to propose a formulation of pure dimethoxy curcumin-human serum albumin which has stability resulting in more cytotoxicity as compared to free DMC, on fibroblast cell line.

A further object of this disclosure is to propose a formulation of pure dimethoxy curcumin-human serum albumin which gives high concentration of water soluble DMCHSA upon dissolving the freeze dried powder unlike poor solubility of dried DMC powder and the water dissolved DMCHSA does not undergo quick metabolic breakdown unlike free DMC.

A still further object of this disclosure is to propose a formulation of pure dimethoxy curcumin-human serum albumin which is transported across cell membrane to result in the biologic actions on cells.

Another object of this disclosure is to propose a formulation of pure dimethoxy curcumin-human serum albumin which is non cytotoxic at low dose and cytotoxic at higher dose.

Yet another object of this disclosure is to propose a formulation of pure dimethoxy curcumin-human serum albumin which can act as an anti-inflammatory drug at low nontoxic dose.

A further object of this disclosure is to propose a formulation of pure dimethoxy curcumin-human serum albumin which can induce death of breast and lung cancer cells in a dose dependent manner.

A still further object of this disclosure is to propose a formulation of pure dimethoxy curcumin-human serum albumin which has intravenous safety without causing haemolytic effect on red blood cells and has intravenous compatibility.

Another object of this disclosure is to propose a formulation of pure dimethoxy curcumin-human serum albumin which can be eliminated through renal route after reasonable residence time of 24h to 48h reducing the incidence of systemic toxicity.

Accordingly, the present disclosure provides a formulation of pure dimethoxy curcumin-human serum albumin (DMCHSA) comprising a pure di-methoxy curcumin (DMC) bound to human serum albumin (HSA), wherein molar ratio of DMC to HSA is in the range of 3.0-6.0.

In one of the feature of the present disclosure, DMCHSA is transported across cell membrane into cytoplasm with significantly higher cytotoxic effect as compared to DMC in fibroblasts, cytotoxic to cancer cells and primary cells, and triggering anti-inflammatory effects on primary human cells. In yet another feature of the present disclosure, the di-methoxy curcumin is highly water soluble, stable, and hemocompatible.

In another feature of the present disclosure, the formulation has the molar ratio of DMC to HSA is in the range of 3.0-4.0.

In one of the feature of the present disclosure, the DMCHSA is non-toxic in a dose range of 1 mg to 15 mg curcumin per kg body weight administered intravenously in mice and the DMCHSA upon entering cell cytoplasm is nontoxic at low dose range and cytotoxic at higher dose to both cancer cell lines and primary human cells.

The present disclosure also provides a process for preparing formulation of pure dimethoxy curcumin-human serum albumin (DMCHSA) wherein said process comprises the steps of:
(i) stirring a pharmacopoeia grade commercially obtained solution containing 1 g to 2 g human serum albumin (HSA) per 5 ml to 10 ml saline;
(ii) adding 10-12 mg of curcumin mixture/curcuminoids comprising 80 to 95 % of di-methoxy curcumin dissolved in 100 µl dimethyl sulfoxide (DMSO) to the solution of step (i) slowly in the amount 5ul at time under gentle mixing to obtain a mixture;
(iii) incubating the mixture of step (ii) for 1 hour to 2 hour at 20° C. to 24° C. to obtain a conjugate; and
(iv) eluting the conjugate using molecular sieving process to obtain a DMCHSA.

In yet another feature of the present disclosure, wherein the above process, curcumin mixture/ curcuminoids is added in amount of 12 mg or 10 mg per 1 g HSA in 5 ml and the 10 mg reduced waste of DMC in the unbound form. The Curcumin mixture/ curcuminoids contains 3 types of curcuminoids, mainly di-methoxy Curcumin (DMC), Demethoxycurcumin (DeMC) and Bidemethoxycurcumin (BiDeMC).

In one of the feature of the present disclosure, the process comprises the steps of:
(i) stirring the pharmacopoeia grade commercially obtained solution containing HSA in the saline;
(ii) adding a 0.4 M - 0.6 M solution of mixed curcuminoid containing di-methoxy curcumin (DMC), demethoxycurcumin (DeMC) and Bidemethoxycurcumin (BiDeMC)and dissolved in DMSO, to the HSA solution;
(iii) adding curcuminoid containing dimethoxy curcumin solution in an aliquot of 0.0001 to 0.0002 vol of HSA at a time;
(iv) making up the final curcumin to HSA proportion of 12:1000 in mg to obtain a solution;
(v) mixing the solution continuously for 1-2 hour at a temperature of solution kept between 20° C. to 24° C.;
(vi) centrifuging the mixture of step (v) at 1000 g to 2000 g for 5-10 min to settle the particulate (insoluble) of excess un dissolved curcumin, comprising DMC, and other contaminating curcuminoids;
(vii) decanting the supernatant to a fresh tube removing precipitated curcuminoids and injecting the clear solution to a column packed with Sephadex G25;
(ix) separating DMCHSA from free curcuminoids by size exclusion principle and pooling eluted fractions of DMCHSA with high absorbance at 280 nm and 420 nm.

In yet another feature of the present disclosure, wherein the above process, the DMCHSA of step (ix):
(a) pass through porous membranes of 0.22 µm porosity for sterilization and estimating the actual concentration of the curcumin per unit volume of solution using spectrophotometry;
(b) dispensing into small vials achieving defined DMC content in each vial and Freeze-drying the product in the vial into dry powder form and vacuum sealing and storing between 2-8° C.; and
(c) dissolving the dry powder in water to obtain soluble DMC to a maximum of 2.0 mg ml⁻¹.

In yet another feature of the present disclosure, preferential binding of DMC to HSA excludes other curcuminoids such as Demethoxy curcumin (DeMC) and Bidemethoxy curcumin (BiDeMC) present in the added raw chemical/ curcumin mixture/ curcuminoids, increasing the purity of 80% DMC in starting raw material to >99% in the product DMCHSA.

In yet another feature of the present disclosure, the process demonstrates to increase dimethoxy curcumin (DMC) purity through selective conjugation to albumin. The less hydrophobic curcuminoids such as demethoxy curcumin (DeMC) and bi-demethoxy curcumin (BiDeMC) present in an impure mixture of commercially available curcuminoid raw material and excess unbound DMC were removed from the reaction mixture by molecular sieving principle

In yet another feature of the present disclosure, the stability of the pure product of DMCHSA was increased by freeze drying into a powder form enabling long term storage.

In another feature of the present disclosure, the pure DMCHSA aiding 600 times more solubility than free DMC.

In still another feature of the present disclosure the DMCHSA preventing hydrolytic degradation of DMC when stored as lyophilized powder and after dissolving back in water.

In still another feature of the present disclosure, the biologically active DMCHSA the native conformation of HSA is maintained allowing receptor recognition and transport of DMC across cell membrane into cytoplasm.

In yet another feature of the present disclosure the DMCHSA showing significantly high cytotoxic effect as compared to free DMC on fibroblast cell lines claiming better stability and function of the product in aqueous medium.

In yet another feature of the present disclosure, the biologically active DMCHSA at lower non-cytotoxic dose range is showing anti-inflammatory property.

In yet another feature of the present disclosure, the biologically active DMCHSA at higher cytotoxic dose range is showing anti-cancer effect.

In yet another feature of the present disclosure, the biologically active DMCHSA is hemocompatible and safe for intravenous administration.

In yet another feature of the present disclosure, the biologically active DMCHSA that circulates in the whole body upon intravenous administration making it bioavailable in all organs for treatment of diseases like inflammation or cancer.

In still another feature of the present disclosure the DMCHSA circulate in whole body eliminates through renal route reducing chances of long-term toxicity.

### BRIEF DESCRIPTION OF THE ACCOMPANYING DRAWINGS

The disclosure may be more fully understood by reading the following detailed description of the embodiment, with reference made to the accompanying drawings as follows:

FIG. 1 shows a summary of the process of conjugate preparation, with FIG. 1A showing stirring/mixing of raw materials-HSA and 80% pure curcuminoid obtained from Sigma Chemicals, USA; FIG. 1B showing centrifuged settled Curcumin; FIG. 1C shows decanted fluid; FIG. 1D shows column chromatography; FIG. 1E shows aliquots; and FIG. 1F shows lyophilized DMCHSA;

FIG. 2 depicts data showing selective binding of and elimination of unbound curcumin or its derivatives, with FIG. 2A showing FTIR of DMCHSA Raw material; FIG. 2B demonstrating FTIR of HSA-bound curcumin eluted from column; FIG. 2C showing unbound curcumin eluted from column; and FIG. 2D showing unbound curcumin derivatives with different chemical characteristics washed out from column using 30% ethanol;

FIG. 3 illustrates stability of DMCHSA as compared to fast degrading free curcumin, with

FIG. 3A illustrating UV-Vis Spectral Properties of degrading free curcumin in 2h; FIG. 3B. illustrating overlaid UV-Vis spectra showing stable nature of DMCHSA 0h-48h period; FIG. 3C showing FTIR spectrum of Fresh DMCHSA; FIG. 3D showing FTIR Spectrum of 24h stored DMCHSA; FIG. 3E showing FTIR Spectrum of 48h stored DMCHSA; and FIG. 3F showing FTIR spectrum of 72h stored DMCHSA;

FIG. 4 illustrates increasing stability of DMCHSA upon storing lyophilized samples, with FIG. 4A showing FTIR spectrum of 6 m stored DMCHSA; FIG. 4B, Functional stability of 9 m stored DMCHSA showing dose dependent cytotoxic effect on cancer cell line A549; FIG. 4C, HPLC elution profile of raw curcumin; FIG. 4D, HPLC elution profile of curcumin extracted from fresh DMCHSA; and FIG. 4E, HPLC elution profile of DMC eluted from DMCHSA stored for 9 months.

FIGS. 5A-5C illustrate raw material independent conjugation of curcumin to has, with FIG. 5A showing overlaid NMR spectra comparing 3 products of DMCHSA prepared using 3 different curcumin; FIG. 5B showing overlaid FTIR spectra comparing 3 products of DMCHSA prepared using 3 different curcumin; and FIG. 5C depicting a graph comparing 3 products of DMCHSA prepared using 3 different curcumin for dose dependent cytotoxic effect on A549.

FIGS. 6A-6C illustrate cell specific transport of DMCHSA into cytoplasm across cell membrane, with FIG. 6A showing endocytosis into A549; FIG. 6B showing endocytosis into endothelial cells; and FIG. 6C showing endocytosis into chrondrocytes.

FIGS. 7A-7D illustrate cell-specific cytotoxic effect, with FIG. 7A showing a graph illustrating dose dependent cytotoxic effect of DMCHSA on A549; FIG. 7B showing a graph illustrating dose dependent cytotoxic effect of DMCHSA on MCF-7; FIG. 7C showing a graph illustrating dose dependent cytotoxic effect on EC; and FIG. 7D showing a graph illustrating dose dependent cytotoxic effect on chondrocytes.

FIG. 8 depicts a demonstration of functional stability of DMCHSA as compared to free DMC in fibroblasts (L929 mouse myeloma cell lines), with the data showing significant increase in the numbers of viable fibroblasts in 24 h-48 h when treated with free curcumin (FIG. 8B) as compared to when treated with DMCHSA (FIG. 8A) indicating that free curcumin is less cytotoxic and permit cell proliferation increasing the numbers of viable cells in 24 h to 48 h.

FIG. 9 illustrates anti-inflammatory effect of DMCHSA, with FIG. 9A showing a graph illustrating down regulation of inflammatory markers upon treating TNF-α activated EC with DMCHSA; and FIG. 9B showing a graph illustrating down-regulation of inflammatory markers upon treating TNF-αactivated chondrocytes with DMCHSA;

FIGS. 10A-10B show blood compatibility and non haemolytic nature of DMCHSA, with FIG. 10A showing control blood smear stained showing normal RBC morphology; and FIG. 10B showing DMCHSA treated blood smear stained showing normal RBC morphology;

FIG. 11 depicts pharmacokinetics showing absorption, distribution, metabolism and elimination (ADME) of intravenous administered ADME in mice model, with FIG. 11A showing fluorescent images of DMCHSA administered to mice in 1 h to 48 h period showing whole body distribution; FIG. 11B showing accumulation of DMCHSA in liver in 24 h; FIG. 11C showing accumulation of DMCHSA in liver after 48 h; and FIG. 11D showing fluorescent images of urine sample demonstrating elimination in urine in 1 h to 96 h.

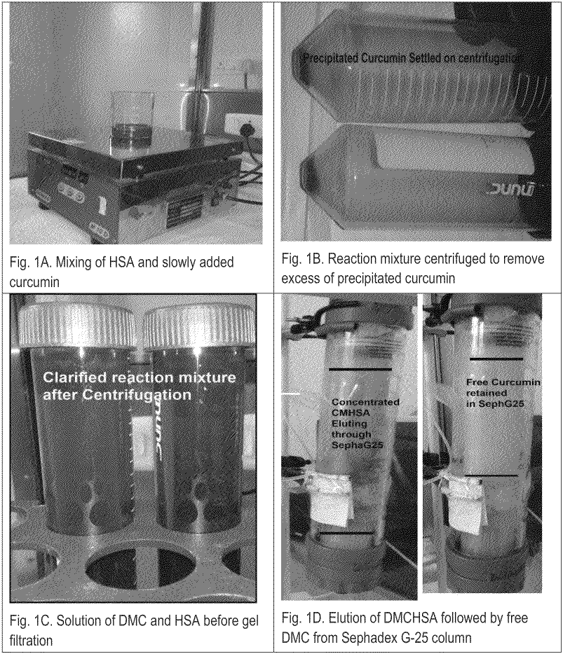

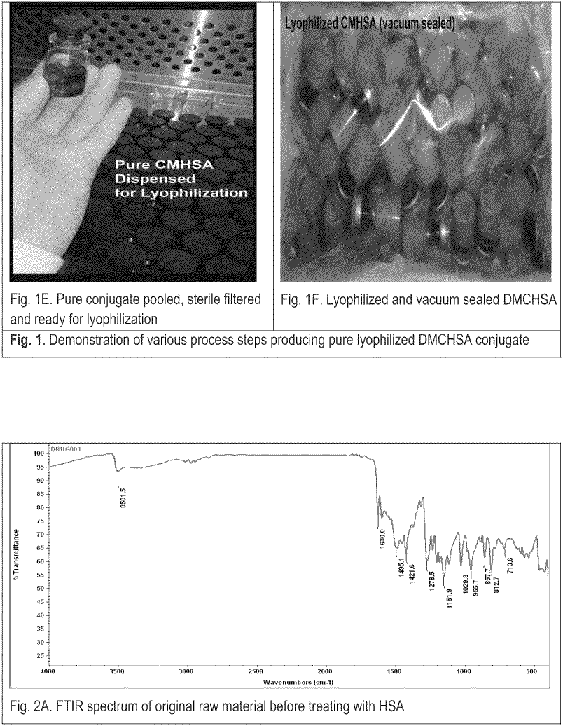

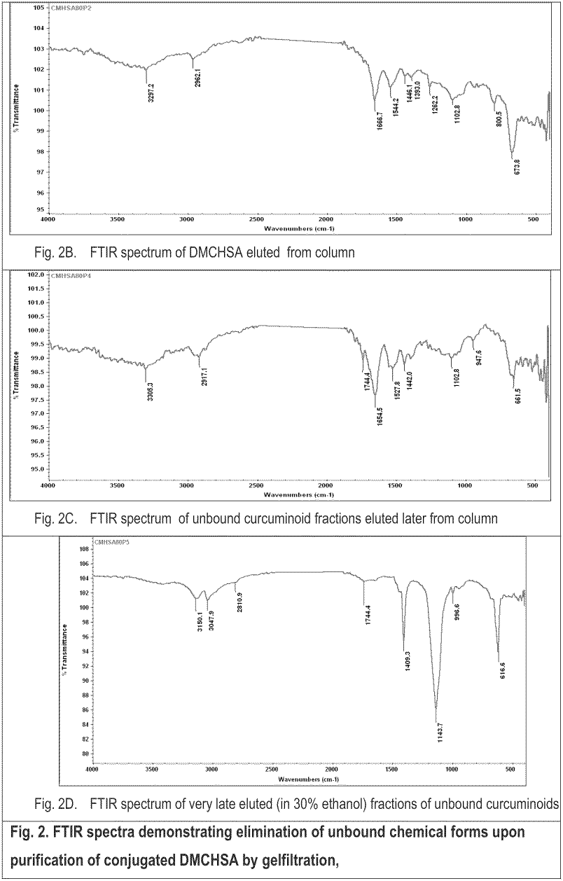

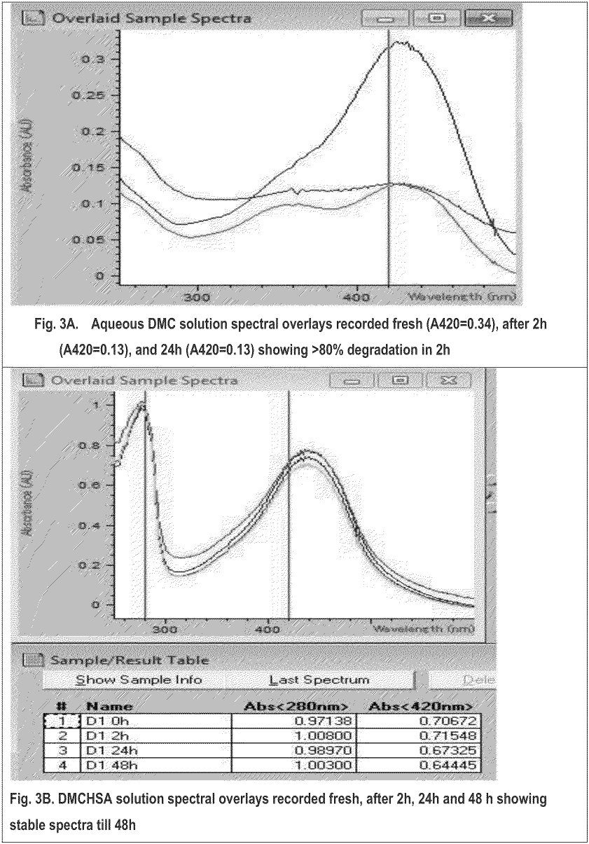

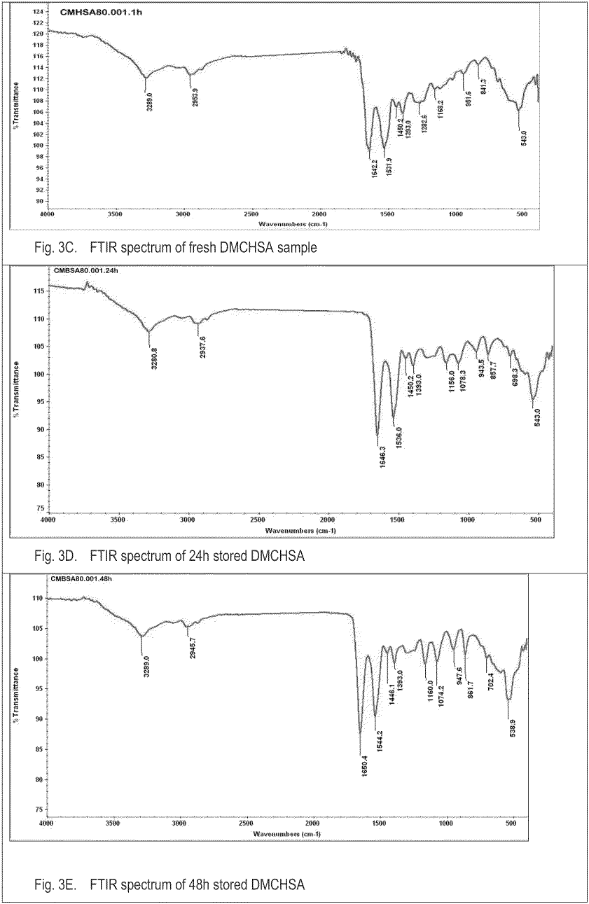

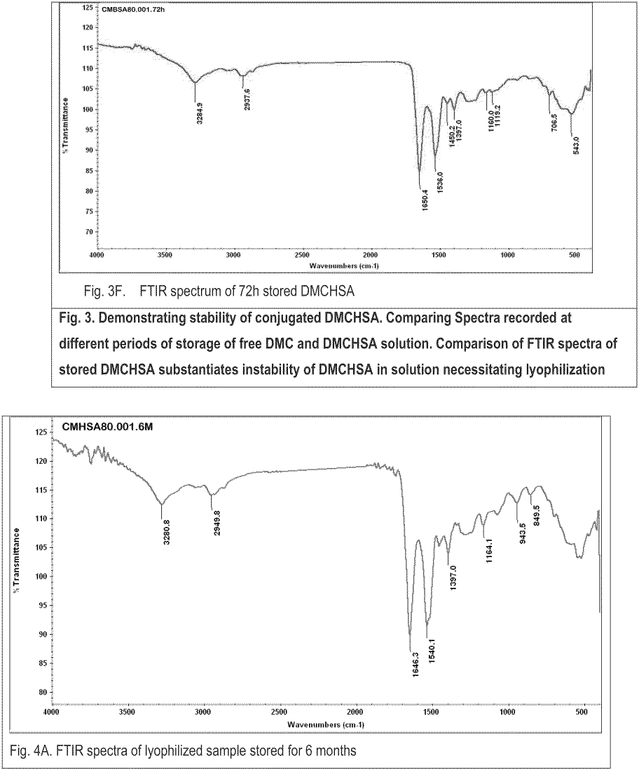

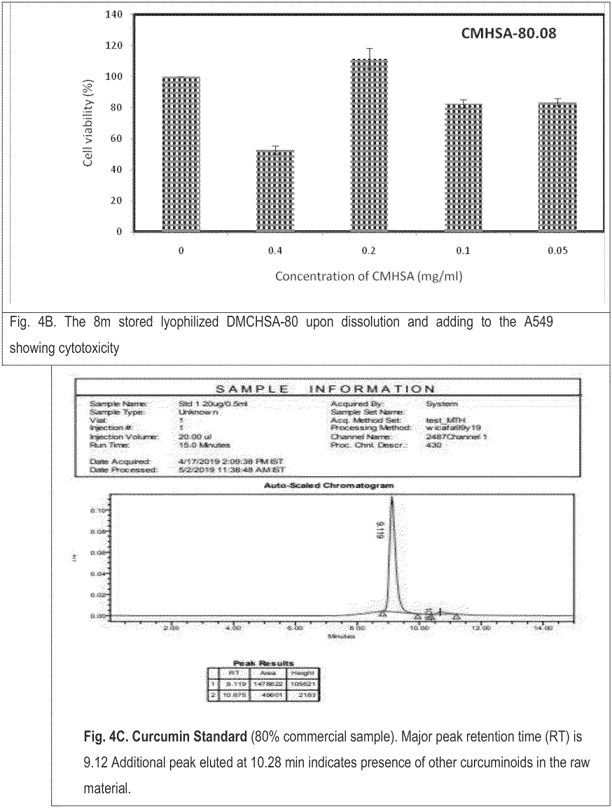

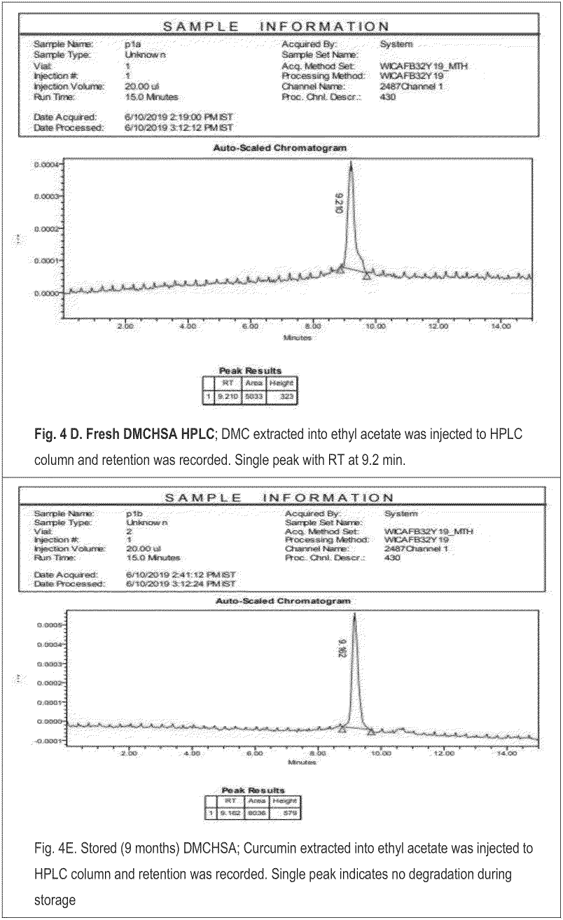

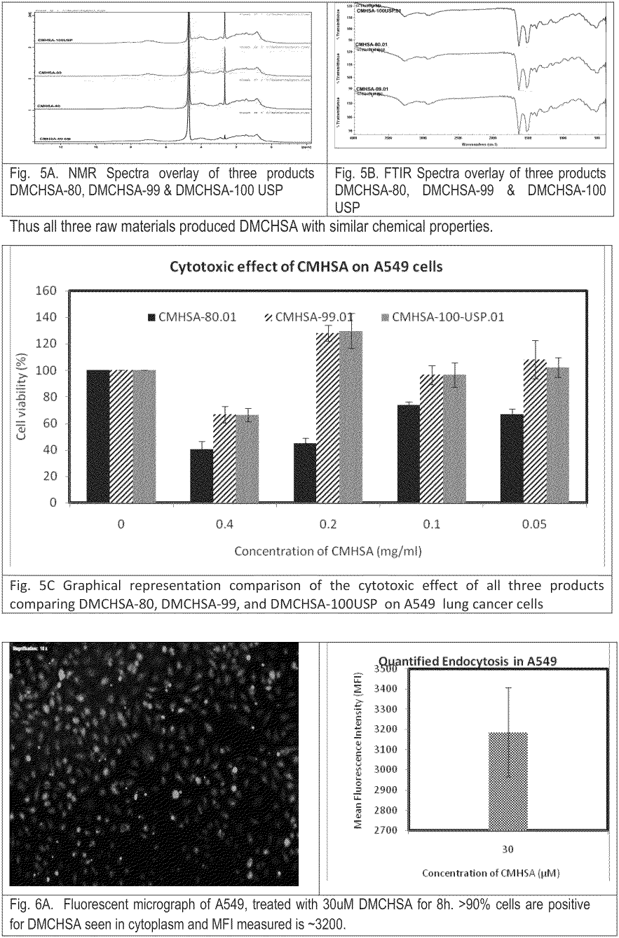

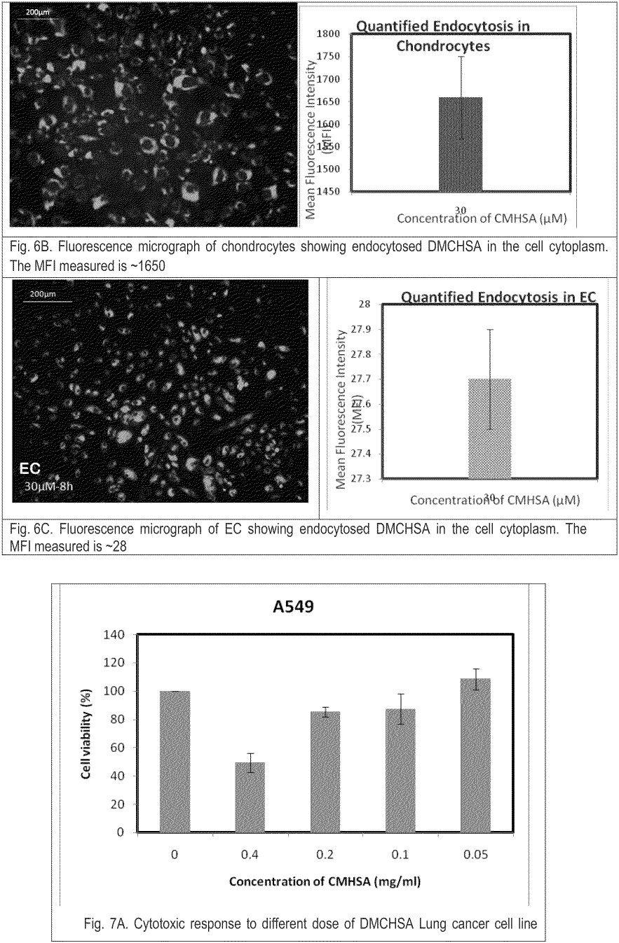

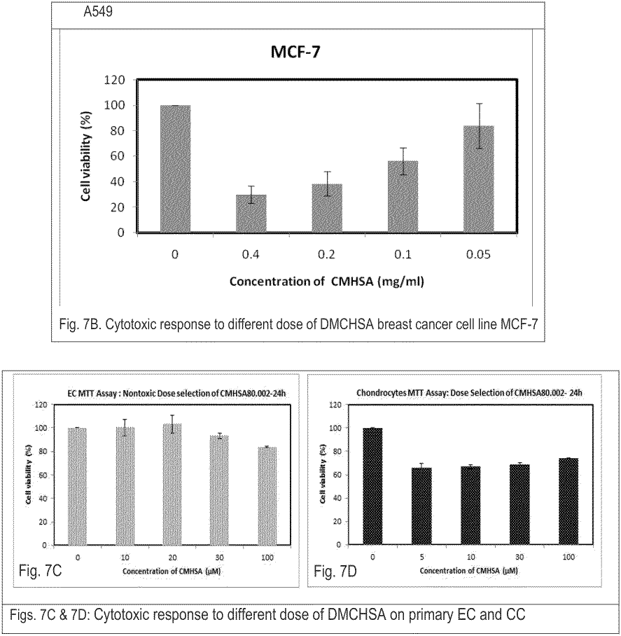

### DETAILED DESCRIPTION

According to this disclosure, there is provided a formulation of pure dimethoxy curcumin-human serum albumin (DMCHSA) and a process for the preparation thereof.

In accordance with one or more embodiments, the formulation of pure dimethoxy curcumin-human serum albumin (DMCHSA) is a highly soluble and safe intravenous formulation of pure dimethoxy curcumin-human serum albumin retaining proven biological activities.

The problem was approached in the following manner:
1. Exploit higher hydrophobicity of DMC for its selective binding to HSA.
2. Apply molecular sieving principle to remove unbound curcuminoids from the product
3. Use HPLC analysis to prove homogeneity of the bound molecule in the product
4. Use fluorescent conjugated albumin to prove transport of DMCHSA into cell cytoplasm
5. Use MTT assay to identify cytotoxic response to different cell lines and primary cells
6. Use cultures of cancer cell lines MCF-7 and A549 to determine effect of DMCHSA for inducing cancer cell death
7. Demonstrate stability and higher cytotoxic activity of DMCHSA as compared to free DMC
8. Use cytokine stimulation to generate inflammatory ECs and chondrocytes and test anti-inflammatory effect of DMCHSA.
9. Employ standard ISO10993-part4 for establishing hemocompatibility and intravenous safety of DCMHSA
10. Employ ISO10993 to evaluate systemic toxicity level of DCMHSA upon intravenous administration in mice model.
11. Use mice model to conduct pharmacokinetics (PK) studies determining the absorption, distribution, metabolism and elimination (ADME) of DMCHSA
12. Demonstrate scale up production of DMCHSA using pharmacopoeia grade HSA and curcuminoid containing 80% DMC as raw materials to obtain homogenous product.

In the present embodiment, the ability of HSA to preferentially bind more hydrophobic molecules has been exploited. One or more embodiments demonstrate a process to increase dimethoxy curcumin (DMC) purity through selective conjugation to albumin. The less hydrophobic curcuminoids such as demethoxy curcumin (DeMC) and bi-demethoxy curcumin (BiDeMC) present in an impure mixture of commercially available curcuminoid raw material and excess unbound DMC were removed from the reaction mixture by molecular sieving principle. Human serum albumin (HSA) in pure form as specified in pharmacopoeia suitable for clinical intravenous administration was another major raw material. The stability of the pure product of DMCHSA was increased by freeze drying into a powder form enabling long term storage. Upon dissolving in aqueous medium, high concentrations of DMCHSA for producing different dose range for biological use. Said conjugate has a molar ratio of DMC to HSA in the range of 3.0-6.0, which theoretically permits receptor mediated entry into cells.

According to embodiments, there is provided a highly soluble albumin conjugated form of DMC for prospective application in curing inflammatory diseases and cancer. Said albumin is selected from different commercially available, pharmacopoeia grade human serum albumin (HSA).

In an embodiment, there is provided an albumin DMC conjugate wherein said conjugate has a molar ratio of DMC to albumin is in the range of 3.0 -6.0.

In another embodiment the albumin is pharmacopoeia grade human serum albumin (HSA) safe for intravenous administration.

In yet another embodiment, the molar ratio of DMC to HSA ranges from 3-4 upon selecting 80% curcumin as raw material for conjugation.

In one embodiment, there is provided a process of preparing the albumin conjugate wherein said process comprises the steps of:
Gently stirring a pharmacopoeia grade solution containing HSA in physiological saline (with stabilizers),using a magnetic stirrer
Adding curcumin dissolved in dimethyl sulfoxide (DMSO) to the above solution slowly at time under gentle mixing;
Incubating the aforesaid mixture for about 1 hour at 20° C. -24° C.; and
Eluting the conjugate using molecular sieving principle.

In one embodiment, there is provided a process of preparing the albumin conjugate wherein said process comprises the steps of:
Gently stirring a pharmacopoeia grade solution containing 1000 mg (1 g) HSA per 5 ml physiological saline (with stabilizers),using a magnetic stirrer;
Adding curcumin (10-12 mg) dissolved in 100 µ1 dimethyl sulfoxide (DMSO) to the above solution slowly- 5ul at time under gentle mixing;
Incubating the aforesaid mixture for ~1 hour at 20° C. -24° C.; and
Eluting the conjugate using molecular sieving principle.

In accordance with this embodiment, different concentrations of curcuminoid mixture was added such as 12 mg or 10 mg per 1 g HSA in 5 ml and the binding was comparable; but 10 mg reduced waste of DMC in the unbound form.

While the free DMC metabolizes to other inactive products at physiological pH, the DMCHSA showed several months stability in the lyophilized form. Increased transportation of the DMCHSA formulation across cell membrane, into cytoplasm of cancer cell (A549) as compared to endothelial cells (EC) and chondrocyte is demonstrated and could be receptor-mediated action. Also, aspects of this disclosure demonstrate intake of bound curcumin by cancer cells and inflammatory cells inducing cytotoxicity and down regulation of inflammatory response, respectively.

The dissolved DMCHSA was experimentally demonstrated to cross membrane of various cell types including cancer cell lines, inflammatory primary cells such as endothelial cells and chondrocytes. Low dose range of DMCHSA was found to be non-toxic and at high dose the molecule induced cytotoxicity and produced non-viable cells in culture. The higher cytotoxic effect of free DMCHSA on fibroblast cell line as compared to free DMC indicates that free molecule undergo degradation and is less active. Fibroblasts continued to multiply producing more viable cells in culture in the presence of free DMC, whereas the similar dose of DMCHSA caused cytotoxic effect reducing the numbers of fibroblasts in 24h-48h of culture. Also, at low non-cytotoxic dose, the DMCHSA prevented cytokine induced inflammatory response in endothelial cells and chondrocytes, indicating its potential use for treating inflammation, and thus to prevent atherosclerotic and arthritic diseases. At high dose range DMCHSA turned both breast and lung cancer cells into nonviable phenotype in respective cultures, indicating its potential to treat cancer.

In the in vitro experiments, absence of DMCHSA induced change to RBC morphology (12.5 µg/ml bound curcumin/ml blood) or hemolysis was eliminated upon treating high concentration (0.2 mg bound curcumin/ml blood); thus ensuring intravenous safety.

In vivo animal studies showed the essential distribution of the bound molecule throughout the body, accumulation in liver for further metabolism and elimination through renal route. Being a cytotoxic anti-cancer drug, the pharmacokinetics study carried out suggests adequate distribution and clearance for preventing other normal tissues from excessive cytotoxic damage.

Albumin solution and dissolved curcumin are taken and mixed at high concentration but in low volume of DMSO. The mixture is centrifuged to remove precipitated (undissolved) curcuminoids from the mixture and subjected to molecular sieving to remove un-reacted curcuminoids and unreacted albumin.

The conjugated fractions were identified with the help of diode array spectrophotometer. The spectral peak ratio of albumin (280 nm) and curcumin (420 nm) was used to eliminate fractions of unbound DMC, other curcuminoids and albumin.

The primary objective was to standardize conditions for preparation of DMCHSA complex and to establish purity of the conjugate, yield, stability and activity. Parameters tested were the effect of different raw materials on the binding efficiency and purity. Other aims were to establish conjugation of DMC to HSA by spectroscopic methods, prove stability and solubility of the bound DMC and demonstrate biological activity of the complex on cancer cell lines and primary human inflammatory cells in vitro. Also, scaled up production of DMCHSA was demonstrated indicating commercial value of the product.

### Preparation of Conjugate

Pharmacopoeia grade HSA from three different sources were used to establish reproducibility. All the three sources used are pharmacopoeia grade but produced and marketed under different trade names and all products are standard preparations of 20 g of albumin in 100 ml (200 mg/ml) containing 123.5-136.5 mM NaCl, 16 mM Acetyltryptophan and 16.0 mM Sodium Caprylate. The HSA solution was withdrawn from the original container using a syringe and needle. The solution was transferred to a small beaker and kept stirring on a magnetic stirrer. From a 0.5 M stock in DMSO, small aliquots of curcumin were added to HSA, with continuous stirring for 1 h, maintaining the temperature between 20° C.-24° C. The solution was then centrifuged for 10 min at 1000 g, to settle the particulate (insoluble) of excess undissolved DMC, and other contaminating curcuminoids. The clear but deep yellow supernatant was then decanted and injected to gel filtration column packed with Sephadex G-25 beads and pre-equilibrated with physiological saline. The peak that eluted the conjugate was identified by absorbance measurement in the first elution peak. The fractions which showed minimum A280/A420 ratio were pooled and used for further evaluations. All fractions with <2 ratio was pooled, sterile filtered using 0.22 µm Millipore syringe filter and dispensed. The column was regenerated for next run by washing with 3 bed volumes 30% ethanol and re-equilibrating with saline.

### Conjugate Characterization

Characterization of conjugated product was carried out with UV-visible absorption spectroscopy (Diode array spectrophotometer, Hewlett Packard 8453), infrared (Jasco 6300 FT-IR spectrometer) /Raman spectroscopy (Bruker RFS 100/s FT-Raman spectroscope).

### Detection of Curcumin Concentration in 1 Ml Aliquot

Purified, pooled conjugate was dispensed into 1 ml fractions and lyophilized. To extract the bound curcumin, 0.1:9 mixture of water DMSO was added, vortex mixed, centrifuged to remove the protein debris and the absorbance of the supernatant was measured at 420 nm (Max absorption of curcumin) in a diode array spectrophotometer. For quantification of the extracted curcumin, a standard curve was prepared using serially diluted curcumin in DMSO. Molar concentration of curcumin in 1 ml was estimated based on the MW of 368.

### Detection of Albumin in 1 Ml Aliquot

To 1 vial of lyophilized DMCHSA, 1 ml of water was added. After complete dissolution the protein concentration was estimated using Lowry’s method. Molar concentration of Albumin in 1 ml conjugate was estimated based on the MW of 66000.

Binding Ratio: The ratio of curcumin to albumin was estimated based on the molar concentration of each constituent in the conjugate.

### Raw Material Validation

Commercially available curcumin (80% or 99% or USP grade Sigma-Aldrich, USA), identified by different catalogue numbers and pharmacopoeia grade human serum albumin (Intas Pharmaceutical Ltd India) were used. Objective was to demonstrate that irrespective of the purity of the curcumin, bound form attained homogenous chemical form of curcumin i.e. dimethoxy curcumin, and all products showed similar FTIR spectra and NMR spectra. In the unbound form which eluted in the later fractions of the column washing contained other chemical forms of curcumin or degradation products are detected. The conjugate chemical characteristics is found to be independent of the raw material (curcumin) used for reaction. Both overlaid NMR spectra and overlaid FTIR spectra of the products prepared using different grades of commercially available curcumin is demonstrated.

### Identification of Excluded Impurities

In order to prove other impurities in the raw material curcuminoid are excluded from DMCHSA, upon binding followed by size exclusion chromatography, the following experiment was done.

The reaction mixture for preparing DMCHSA contained 200 mg HSA and 2.88 mg C7727. The mixture was subjected to molecular sieving chromatography on 25 ml Sephadex G-25 column. Five ml fractions were collected and were analysed by both A280 and A420 absorbance. Fractions with highest HSA content, medium HSA content and very low HSA content but with high DMC absorbance were pooled and designated as P1, P2, P3 respectively. After completing elution with 2 bed volume of saline, another 100 ml was collected as Pool4 (P4). Once the column wash volume completed 150 ml, (i.e. 6× bed volumes), still the column shows yellow colour. Therefore, the column was washed with 50 ml of 30% ethanol and the eluted fractions were collected as pool5 (P5).

Both Pools 4 and 5 were lyophilized and FTIR spectrum was recorded and compared with raw material as the drug001 as sample for comparison. The FTIR spectrum of DMCHSA in P2 with 280 nm and 420 nm absorbance also was also recorded.

The different chemical characteristic of the unbound curcumin was established by FTIR spectroscopy analysis of the pooled fractions of later eluted unbound chemical. Comparison of the original raw material, conjugated DMCHSA and unbound chemicals are demonstrated.

### Storage Stability of DMCHSA Solution

The liquid stability of the product was analysed at 24 h interval for 48 h or 72 h. The UV spectra recorded at different storage period are shown

FTIR spectra are shown to substantiate the chemical degradation after 48 h of storage of DMCHSA solution.

From both analysis it is confirmed that DMCHSA solution showing minor degradation after 48 h.

All liquid stored samples were analysed for functional efficacy in terms of cytotoxicity to lung cancer cells A549.

Sample Preparation:
Freshly prepared DMCHSA-80.01
24h liquid stored DMCHSA-80.01
48h liquid stored DMCHSA-80.01

A549 cells (2.5×10³) were seeded into each well of 96 well plates. The cells were allowed to grow till it appeared nearly confluent. Standard MTT assay was carried out to determine the cytotoxic effect of DMCHSA-80.01. Functional stability up to 48 h of liquid storage is confirmed.

Therefore, lyophilisation immediately after preparation of DMCHSA as normal practice is found essential.

### Storage Stability of Lyophilized DMCHSA

The lyophilized samples were used for respective periods as prepared for liquid stability, were lyophilized and recorded FTIR spectrum. The spectral properties are compared Spectral characteristics showed noticeable difference in the liquid sample stored for 72 h.

Thus, liquid stability of DMCHSA stored for 48 h is evident.

The lyophilized sample stored in 2-8° C. for 6 m to 8 m was verified for the chemical and functional stability and the results are demonstrated.

### Transport of DMCHSA Across Cell Membrane

The cytotoxic effect could vary depending on the number of molecules entering the cells and the final concentration achieved in the cell cytoplasm. Therefore, transport of DMCHSA across cell membrane (endocytosis) was measured in different cells such as cancer cells (A549), endothelial cells (EC) and chondrocytes (CC) and compared. As expected, receptor mediated endocytosis of DMCHSA could take place in all cells.

To track endocytosis, two aliquot of DMCHSA-80 (total 0.4 mg Curcumin bound to 50 mg albumin) was dissolved in carbonate buffer pH 9.0 and tagged with FITC (50 mM) using standard method. Unreacted FITC was removed by gel-filtration on Sephadex G-25. Fractions with >2.0 A495:A280 ratios were pooled, sterile filtered (0.22 µm) and lyophilised as 0.2 ml aliquots for tracking experiments. The curcumin from aliquots of FITC-tagged DMCHSA were extracted into 9:1 DMSO-water mixture and curcumin was quantified based on standard curve.

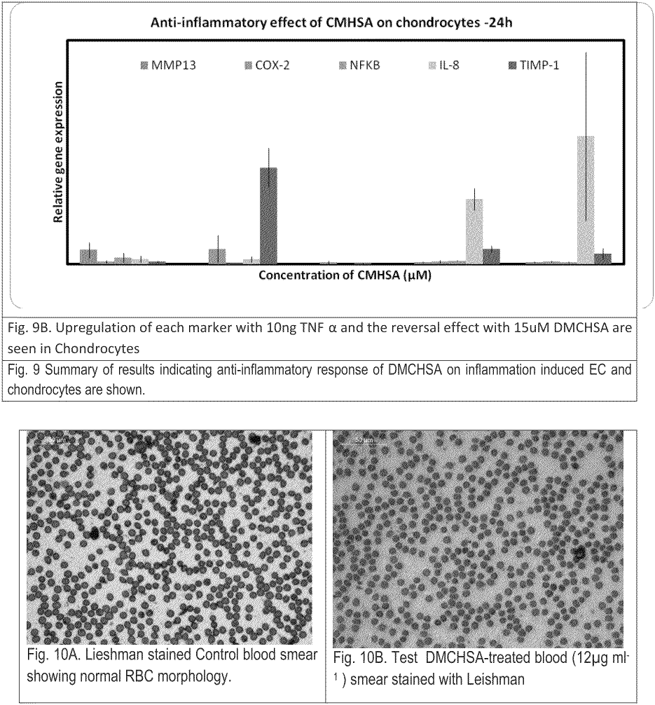

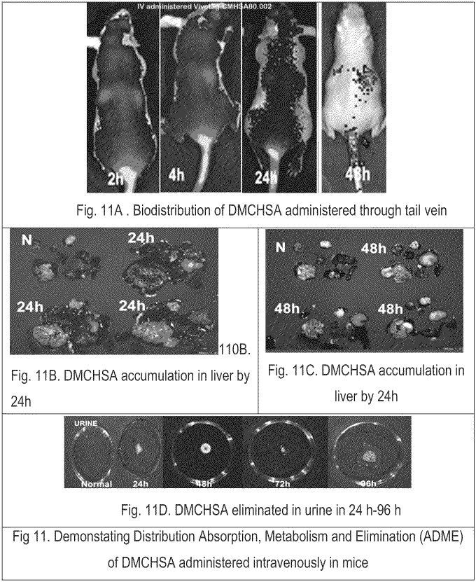

For analysis of DMCHSA endocytosis, A549, EC and CC cultures (>70% confluent) were treated with 30 uM concentration of FITC-DMCHSA. The cultures were allowed to grow under standard conditions. The cells were analysed using fluorescence microscope (Leica system) and the fluorescent images were captured using LAS camera and software. The cells were harvested by trypsinization and was analysed for fluorescence quantification using FITC channel. The mean fluorescence intensity of 4 replicate culture samples were computed to get average MFI in each case (FIGS. 9A-9D)

The results indicated that while > 90% cells endocytosed DMCHSA in 8 h, the numbers of molecule entering the cell is different depending on the cell type, in turn increasing the mean fluorescence intensity (MFI) upon flow cytometry analysis. The MFI in A459 is double that of CC and ~10 fold higher than that in primary human EC. Accordingly, cytotoxic response of primary EC was also lower as compared to chondrocytes or to the cancer cells- A549.

Thus the cytotoxicity to A549>Chondrocytes>Endothelial cell has been established

### Cytotoxic Effect of DMCHSA in Cell Lines and Primary Cells

For cytotoxicity assay, both lung cancer cell line A549 and breast cancer cell line MCF-7 were grown under standard culture conditions. Once the cells achieved 70-80% confluence, cell viability assay was carried out in A549, MCF-7, ECs and CCs using graded concentration of conjugate for a period of 24 h.

Cytotoxicity was assayed by employing the standard MTT assay. This is a colorimetric assay that measures the percentage of metabolically active cells. The test is based on the principle that MTT enters the cells and passes into the mitochondria where it is reduced to an insoluble, coloured (dark purple) formazan product. Reduction of yellow MTT in the reagent by mitochondrial succinate dehydrogenase is the first step. This formazan production is directly proportional to the viable cell numbers and inversely proportional to the degree of cytotoxicity. The formazan are then solubilized with an organic solvent (e.g. DMSO) and the released, solubilised formazan reagent is measured spectrophotometrically. Since reduction of MTT can only occur in metabolically active cells the level of activity is a measure of the viable cells. Thus the metabolic activity of each culture treated with different dose of DMCHSA was determined by the standard MTT assay and compared to those of untreated cells.

Cancer cell lines (A549,MCF-7), Fibroblast cell line (L929), and primary cells (ECs and CCs) were cultured in 96-well plates containing 100 µl medium, kept this plate in CO₂ incubator (5% CO₂) at 37° C. for 24 h. DMCHSA solution was freshly prepared by dissolving the lyophilized conjugate in DMEM F12 medium and added (0.05 mg/ml, 0.1 mg/ml, 0.2 mg/ml, 0.4 mg/ml) in 96 well plate for 24 h. The cytotoxicity of DMCHSA was compared with equivalent concentrations of free DMC by adding similar molar concentrations of the solutions into the fibroblast culture medium. In all cultures standard MTT assay quantified viable cells after treating with DMCHSA/free DMC. Briefly, commercial MTT (0.5 mg/ml) reagent was dissolved in medium. This solution was filtered through a 0.22 µm filter and stored at 2 - 8° C. After removal of 100 µl medium, MTT dye solution was added and the plates were incubated at 37° C. for 4 h in a humidified 5% CO₂ incubator, followed by 100 µl of DMSO added to each well, and mixed thoroughly to dissolve the dye crystals.

The absorbance was measured using an ELISA plate reader at 570 nm. High optical density readings corresponded to a high intensity of dye colour. The fractional absorbance was calculated by the formula: % Cell survival/PR = Mean absorbance in test × 100 Mean absorbance in control

Cytotoxicity of DMCHSA on cells was calculated as cell growth inhibition rate (IR),IR = 100-PR. Compiled results are presented in graphs. The effect on MCF-7>A549>CC>EC in dose dependent manner is demonstrated.

### Anti-Inflammatory Response of DMCHSA

The endothelial cells (ECs) are an important structural and functional component of blood vessels and help in maintaining the integrity of vessel walls. When these cells are exposed to harmful and toxic components they begin to show the after effects by secreting certain cytokine molecules or by expressing cell adhesion molecules on their surface. This sudden change in the cell behaviour is referred to as the endothelial dysfunction and it occurs when the cells are under stress. These stress conditions contribute to the development of atherosclerosis which is marked by the deposition of cholesterol molecules beneath the wall of blood vessels. Slowly as the disease progresses, the blood vessel narrows and hardens resulting in obstructed blood flow making the target tissue which was being supplied with this same vessel devoid of oxygen and nutrients. As time passes the tissue begins to deteriorate and integrity gets destabilized. So, all these events involve immense role played by various cells of the immune system as well as inflammatory mediators, which are either produced by the ECs itself or act on ECs to initiate inflammation.

Another major disease is arthritis which is marked by inflammation of the cartilaginous tissue present at the joints. It is a major causative of movement impairment and also sometimes may lead to mental depression also. The initial events involve cartilage injury whose causes vary, followed by the action of inflammatory cytokines which worsen the situation. Chondrocytes help in the secretion of extracellular matrix (EDMC) that provides the required strength for cartilage. These cells are embedded in the matrix itself in specialized pouches called lacunae. But once there is an insult to the tissue, matrix undergoes biochemical and pathophysiological changes that disrupts the integrity of cartilage.

To evaluate the effect of DMCHSA, inflammatory response in ECs/chondrocytes was established using inflammatory inducer-Tumour necrosis factor-α (TNF-α). Near confluent stage of EC monolayers or CC were incubated with graded concentrations of cytokines: TNF-α (10 ng/ml) for 24 h; the expression of inflammatory markers: Nuclear factor-kB (NF-kB), monocyte chemo-attractant protein-1 (MCP-1), endothelin-1 (ET-1), cyclooxygenase-2 (COX-2), vascular cell adhesion molecule (VCAM-1) were determined to be up-regulated by Quantitative Real Time polymerase chain reaction (qRT-PCR). The expression of the following inflammatory markers: Nuclear factor-kB (NF-kB), cyclooxygenase-2 (COX-2), interleukin-8 (IL-8), Matrix metalloproteinases -13 (MMP-13) and Tissue inhibitor of MMPs (TIMP-1) were determined by qRT-PCR in chondrocytes. The cytokine-induced up regulation of inflammatory markers were calculated against normal ECs/CCs and using Glyceraldehyde 3-phophate dehydrogenase (GAPDH) as house-keeping gene. For analysing the effect of DMCHSA on such activated ECs, initially 24 h exposure of >70% confluent ECs to TNFα (10 ng /ml) was done. Further, the activated ECs/CCs were treated with different dose of DMCHSA (5 to 60 µM) for an additional 24 h. qRT- PCR was carried out as described above to estimate the effects of DMCHSA on the marker expressions.

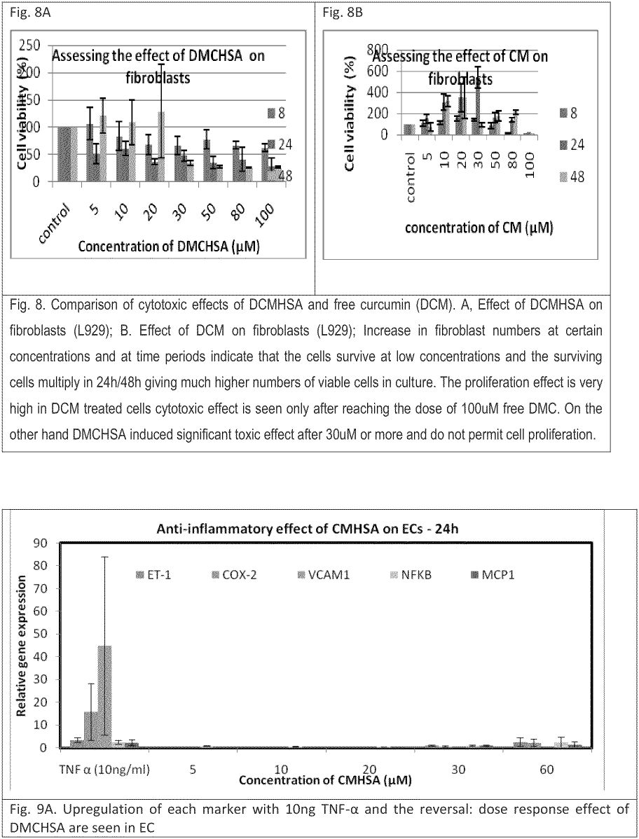

The cell culture experiments thus have proven the anti-inflammatory activity of DMCHSA. A biphasic effect is seen which is due to increased cytotoxicity at higher dose. This is because cytotoxic effect can also up regulate inflammatory genes. Therefore, to achieve anti-inflammatory effect, the dose of DMCHSA should be selected carefully (FIGS. 8A-8B).

### Intravenous Safety

Safety of DMCHSA for intravenous infusion is another aspect. As part of primary qualification of DMCHSA for intravenous applications, the hemocompatibility is an important parameter to be studied.

The test is done as per ISO10993-part4 which is recommended for blood contacting materials and biomedical devices. Human blood is collected using 3.8% citrate as anticoagulant. An aliquot of blood is centrifuged and plasma haemoglobin is measured as part of ISO17025 quality system to qualify the blood sample for testing the effect of material on hemolysis and RBC morphology. Whole blood is subjected to total blood count using Sysmex 4500 automated haematology analyzer. Blood smears are prepared for analyzing RBC morphology (control).

One millilitre of anticoagulated human blood was treated with 6.25 µg to 200 µg of DMCHSA for 30 min. Using the whole blood, smears were prepared and stained (Lieshman’s) for observing RBC morphology (Test) under microscope to compare control RBCs with test RBCs.

Blood was centrifuged at 1000 g for 10 min. Platelet poor plasma is aspirated and subjected to spectrophotometric detection of plasma hemoglobin. Based on the plasma hemoglobin, the % hemolysis was calculated. % hemolysis= (plasma hemoglobin/total hemoglobin in whole blood) ×100

No morphological change to RBC was observed upon treating blood with 12.5 µg, some cells with slight crenations were seen without any lysis were seen above 25 µg/ml blood No other abnormal change in morphology were noted in all concentration studied. The % haemolysis was <0.1% at a concentration of 200 µg /ml blood, which is similar to normal blood. The concentration (0.2 mg ml⁻¹) is which is much higher than expected therapeutic dose for inducing apoptosis as per the in vitro data.

### Pharmacokinetics in In Vivo Model

Further, the absorption, distribution, metabolism and elimination (ADME) upon intravenous administration of DMCHSA determines the safety and predicts the resident time available for the molecule to localize in the cancer/inflammatory tissues and cells.

For labelling of Vivotag-750 S to DMCHSA, required amount was dialyzed in carbonate buffer (pH 8.5) for 24 h with three exchange of dialyzing fluid. Vivotag-750 was dissolved in DMSO. The required quantity of fluorochrome (300 µg for 1 mg protein) was added slowly as 5 µL aliquots and incubated at 20-25° C. for 2 h. The resultant mixture was purified using Sephadex G-25 columns to remove unreacted Vivotag-750 S and 1ml fractions were collected, analyzed, spectrophotometrically at A280, A420 and A750. Fractions found maximum binding with Vivotag-750 S to conjugate were selected, pooled, aliquots of 0.5ml were stored at 4° C.

All the protocols used for the studies were approved by Institutional animal ethical committee (IAEC-SCT/IAEC-263/February/2018/95-06.04.2018). Briefly, hair of Swiss albino mice was completely removed using approved methods to eliminate auto-fluorescence. All animals were housed in metabolic cages prior to experiment for acclimatization. DMCHSA80.002 labelled with Vivotag-750 S was administered at a dose equivalent (Albumin 0.5 mg/kg and of 8.7 µg/kg) through tail vein injected in normal saline and images were taken at 24 h, 48 h, 72 h and 96 h by Xenogen IVIS Spectrum (Caliper Life Science). A set of animals were euthanized at the same intervals, organs like brain, heart, lungs, liver, spleen, gastrointestinal system and kidney were isolated, washed in saline, blotted on a paper and images were taken. Urine and faecal samples were collected at the same time intervals and imaged for determining the route of elimination.

From the whole animal images, it is evident that, DMCHSA distribution in blood is attained at 2 h administration with significant clearance in 24 h and seen localized in liver at 48 h. Maximum elimination is in urine at 24 h and 48 h, which sustain till 72 h. Trace is seen in faecal matter at 48 h and 72 h.

Therefore, DMCHSA is safe for intravenous transfusion and it is eliminated through the renal route after reasonable resident time in the body for cellular uptake and action.

### Example 1

For setting up reaction; 3 g liquid stable albumin (15 ml of 20 g dL⁻¹ solution) was aspirated aseptically from therapeutic preparation. From 0.5 M stock of curcumin in DMSO 200 ul was added to albumin i.e. (~36.8 mg of curcumin) slowly with gentle continuous stirring. After 1 h, the mixture was gel filtered using Sephadex G-25 packed to get bed volume of 25 ml and equilibrated with sterile filtered normal saline. The chromatography system Akta Prime Plus (GE Health Science USA) with UV detector (280 nm) and associated software was used to run the purification protocol in automatic mode. Five 5ml mixture was injected for each run and 3 such runs were made to purify 15 ml reaction mixture. The eluted protein peak was collected as two ml fractions based on A280. Bound molecule in the elute was identified by measuring absorbance at 280 nm (A280) for protein and 420 nm (A450) for bound curcumin (1). Selected fractions with maximum curcumin-specific and albumin-specific peaks were pooled, filtered (0.22 µm), dispensed into 1ml aliquots and lyophilized.

A calibration curve was created using graded concentrations of curcuminoid solution in DMSO; A425 was measured using quartz cuvette and HP8453 Diode Array Spectrophotometer by selecting software in the “quantification’ mode. The calibration curve was stored for later measurement of concentration of curcumin in DMCHSA.

For measurement of DMC in DMCHSA one lyophilized aliquot was dissolved in 1 ml water, 10 ul was added to 990 ul of DMSO and measured the concentration using the calibration curve and total content in 1 ml was calculated based on which the yield in the pool was calculated.

Albumin in the DMCHSA was measured using a calibration curve stored in the HP8453 system by Lowry’s protein assay method. Standard procedure of dilution is applied if concentration is higher than the range in the calibration curve. Based on the measured protein per ml, yield in the pool is calculated.

Finally, the molar binding ratio was calculated to determine how many molecules of curcumin is bound to 1 molecule of albumin in different conditions of reaction.

| Raw material | Concentr ation of reactants | Reactant volumes | Concentration /ml After purification | Volume of pooled fractions | Total yield | Recovery (%) |
|---|---|---|---|---|---|---|
| Drug 001 | 36.8 mg | 200 ul | 0.4 mg | 66 ml | 26.4 mg | 71.7 % |
| Albumin (Intas) | 3 g | 15 ml | 24 mg | | 1584 mg | 52.8 % |

Molarity of drug in the final product: 0.0048 M
Molarity of Albumin in the final product: 0.0016 M
Binding ratio: 3.0

### Example 2

### DMCHSA-10g Batch

Albumin from Kedrion (pharmacopoeia grade) was used and drug from Sigma Chemicals was used. Fifty ml containing 10 g albumin was withdrawn from the sterile vial and added into a 250 ml beaker. The solution was kept under laminar flow placed in Class 10000 room.

Drug (C7277) dissolved in 1.5 ml DMSO was added in small aliquots into the albumin under continuous stirring, over a period of 1h. Temperature of the reaction mixture was 20° C. -24° C.After adding the entire drug, solution was kept under stirring for another 1h. The reaction mixture was transferred into 2 numbers of 50 ml, sterile centrifuge tubes and were centrifuged at 10000 g for 10 min. The unreacted drug settled at the bottom was discarded after decanting the reaction mixture into a fresh 50 ml tube.

The supernatant was further filtered using 0.45um syringe filter. For the 10 g batch, larger column was packed with Sephadex G-25, bed volume 350 ml.

Program Method was Flow rate 5 ml per min; Injection at 10 ml (Break point 1); Injection volume 50 ml; Fraction collection started at 120 ml (break point 2); Fraction size-10 ml; Total fractions collected 40

The column was run using normal saline in which the beads were equilibrated by passing 1 L of saline 0.50 ml injection loop purchased from GE Health, USA was used for loading DMCHSA into the column.

After sample, elution of pure DMCHSA started after ~ 120 ml of normal saline was passed. The eluted conjugate was collected in 10 ml fractions. All fractions having high protein content (280 nm) and drug content (420 nm) were pooled. Total fractions pooled were 25.The concentration of drug in the conjugate and albumin in the conjugate were determined using the calibration curves. Pooled sample was sterile filtered using 0.22um membrane filtered. The conjugate was dispensed into different vials to contain 1 mg /vial, 3 mg/vial and 5 mg/vial. All vials were subjected to lyophilisation and were closed with rubber stopper under vacuum. The rubber caps were closed tightly by crimping aluminium caps.

| Raw material | Concentr ation of reactants | Reactant volumes | Concentration/ ml After purification | Volume of pooled fractions | Total yield | Recovery (%) |
|---|---|---|---|---|---|---|
| Drug 001 | 120 mg | 1.5 ml | 0.3 mg | 250 ml | 75 mg | 62.5 % |
| Albumin (Kedrion) | 10 g | 50 ml | 16.29 mg | | 4072.5 mg | 40.7 % |

Molarity of drug in the final product: 0.05 M
Molarity of albumin in the final product: 0.015 M
Binding Ratio = 3.3

Inference: Irrespective of the batch size, only 60% to 70% drug was bound to albumin. The binding ratio was consistently between 3.0 to 3.5 when 80% pure drug was used. The unreacted drug was removed from the product efficiently by simple molecular sieving chromatography.

We claim:

1. A formulation of pure dimethoxy curcumin-human serum albumin (DMCHSA) comprising a pure di-methoxy curcumin (DMC) bound to human serum albumin (HSA), wherein molar ratio of DMC to HSA is in the range of 3.0-6.0.

2. The formulation as claimed in claim 1, wherein the molar ratio of DMC to HSA is in the range of 3.0-4.0.

3. The formulation as claimed in claim 1, wherein the DMCHSA is non-toxic in a dose range of 1 mg to 15 mg curcumin per kg body weight administered intravenously in mice and the DMCHSA upon entering cell cytoplasm is nontoxic at low dose range and cytotoxic at higher dose to both cancer cell lines and primary human cells.

4. A process for preparing formulation of pure dimethoxy curcumin-human serum albumin (DMCHSA) wherein said process comprises the steps of:
(i) stirring a pharmacopoeia grade commercially obtained solution containing 1 g to 2 g human serum albumin (HSA) per 5 m1 to 10 m1 saline;
(ii) adding 10-12 mg of curcumin mixture/ curcuminoids comprising 80 to 95 % of di-methoxy curcumin dissolved in 100 µ1 dimethyl sulfoxide (DMSO) to the solution of step (i) slowly in the amount 5ul at time under gentle mixing to obtain a mixture;
(iii) incubating the mixture of step (ii) for 1 hour to 2 hour at 20° C. to 24° C. to obtain a conjugate; and
(iv) eluting the conjugate using molecular sieving process to obtain a DMCHSA.

5. The process as claimed in claim 4, wherein the curcumin mixture/curcuminoids is added in amount of 12 mg or 10 mg per 1 g HSA in 5 m1 and the 10 mg reduced waste of DMC in the unbound form.

6. The process as claimed in claim 4, wherein the curcumin mixture/ curcuminoids comprises di-methoxy curcumin (DMC), demethoxycurcumin (DeMC) and Bidemethoxycurcumin (BiDeMC).

7. The process as claimed in claim 4, wherein the process comprises the steps of:
(i) stirring the pharmacopoeia grade commercially obtained solution containing HSA in the saline;
(ii) adding a 0.4 M - 0.6 M solution of mixed curcuminoid containing di-methoxy curcumin (DMC), demethoxycurcumin (DeMC) and Bidemethoxycurcumin (BiDeMC) and dissolved in DMSO, to the HSA solution;
(iii) adding curcuminoid containing dimethoxy curcumin solution in an aliquot of 0.0001 to 0.0002 vol of HSA at a time;
(iv) making up the final curcumin to HSA proportion of 12:1000 in mg to obtain a solution;
(v) mixing the solution continuously for 1-2 hour at a temperature of solution kept between 20° C. to 24° C.;
(vi) centrifuging the mixture of step (v) at 1000 g to 2000 g for 5-10 min. to settle the particulate (insoluble) of excess undissolved curcumin, comprising DMC and other contaminating curcuminoids;
(vii) decanting the supernatant to a fresh tube removing precipitated curcuminoids and injecting the clear solution to a column packed with Sephadex G25;
(ix) separating DMCHSA from free curcuminoids by size exclusion principle and pooling eluted fractions of DMCHSA with high absorbance at 280 nm and 420 nm.

8. The process as claimed in claim 7, wherein the DMCHSA of step (ix):
(i) pass through porous membranes of 0.22 µm porosity for sterilization and estimating the actual concentration of the curcumin per unit volume of solution using spectrophotometry;
(ii) dispensing into small vials achieving defined DMC content in each vial and Freeze-drying the product in the vial into dry powder form and vacuum sealing and storing between 2-8° C.; and
(iii) dissolving the dry powder in water to obtain soluble DMC to a maximum of 2.0 mg m1⁻¹.

9. The process as claimed in claim 4, wherein preferential binding of DMC to HSA excludes other curcuminoids such as Demethoxy curcumin (DeMC) and Bidemethoxy curcumin (BiDeMC) present in the added raw chemical/ curcumin mixture/ curcuminoids, increasing the purity of 80% DMC in starting raw material to >99% in the product DMCHSA.
